(19) United States
(12) Patent Application Publication
(10) Pub. No.: US 2004/0126878 A1
(43) Pub. Date: Jul. 1, 2004

(54) Method for the preparation of immunologically inert amniotic membranes

(76) Inventors: Maria Ramos, Lisboa (PT); Laura Costa, Lisboa (PT); Angelica Roberto, Portela (PT)

Correspondence Address: LADAS & PARRY, 26 WEST 61ST STREET, NEW YORK, NY, 10023, US

(21) Appl. No.: 10/473,515

(22) PCT Filed: Mar. 27, 2002
(86) PCT No.: PCT/PT02/00005

(30) Foreign Application Priority Data
Mar. 30, 2001 (PT) 102589

(51) Int. Cl. C12N 5/08

(52) U.S. Cl. 435/366

(57) ABSTRACT

The present invention discloses a method for preparation of immunologically inert amniotic membranes using foetal membranes collected from programmed caesareans, under sterile conditions. The immunologically inert amniotic membrane of the invention may be applied to the preparation of a product to be used as a skin substitute in 2nd or 3rd degrees burns, as nerve sleeve to guide regeneration of the peripheral nerves, as cornea graft, in the reconstruction of the bladder and urethras, in the correction of cardiac malformations such as inter-auricular and inter-ventricular communications and in the reconstruction of valvular leaflets.

### INTRODUCTION

The present invention discloses a method for preparation of immunologically inert amniotic membranes using foetal membranes collected from programmed caesareans, under sterile conditions. The immunologically inert amniotic membrane of the invention may be applied to the preparation of a product to be used as a skin substitute in 2_{nd} or 3_{rd} degrees burns, as nerve sleeve to guide regeneration of the peripheral nerves, as cornea graft, in the reconstruction of the bladder and urethras, in the correction of cardiac malformations such as inter-auricular and inter-ventricular communications and in the reconstruction of valvular leaflets.

### BACKGROUND OF THE INVENTION

In the particular case of patients with extensive burns, wound coverage remains a major problem both in the acute and reconstructive stages, due to the shortage of autologous skin donor sites¹. Therefore, successful development of a permanent skin substitute would have an enormous impact on the care of such patients.

In 1910 Davis was the first to report the use of foetal membranes (amnion and chorion) as surgical material in skin transplantation (cited from¹), but without a major success for rejection occurred much in the same way as skin homografts.

Skin is a complex organ. Functionally, it has two layers, the epidermis and the dermis. The first, which provides a bacterial barrier, comprises four layers, namely, the strata basale, the spinosum, the granulosum and the corneum. The dermis, with its rete ridges, supplies the strength and elasticity to the skin².

At the present most burn wounds are best closed as quickly as possible with split thickness autograft. However, this autograft is an imperfect replacement for full thickness skin².

Nevertheless the use of amniotic membrane in surgery was expanded and in the 1940s several authors reported the beneficial role of the amniotic membrane in treating a variety of ocular diseases³. Presently, the amniotic membrane has been recognised as an excellent material for the treatment of certain ocular disorders such as persistent corneal epithelial. defects with ulceration⁴⁻⁶, pterygium⁷ and for conjunctival surface reconstruction ^{5,8}. Beneficial effects in leg ulcers, skin loss in Steven-Johnson syndrome and as temporary skin dressing in burn and surgical wounds have also been reported⁹.

Several authors cultured corneal epithelial stem cells on a matrix of amniotic membrane¹⁰, previously denuded, by removing amniotic epithelium using a combination of trypsin digestion and mechanical scraping^{10,11}, in this way amniotic membrane acts only as a substrate for cell growth¹¹.

The eye has for long been recognised as an “immunologically privileged” site for many of its structures are not vascularized and, thus, the immune cells cannot reach and react against them. On the other hand, trypsin digestion damages the extracellular matrix (ECM), which opens for the possibility of activating collagenases, degradation of the ECM, deposition of fibronectin and scar formation.

The present invention circumvents this side effects leaving the ECM intact.

Many attempts have been made to cultured keratinocytes using various types of matrix like polyurethane membranes¹² or acellular collagen matrices¹³. In recent years, skin grafting has evolved from the initial autograft and allograft preparation to biosynthetic and tissue-engineered skin replacements¹³. Thus, the availability of new materials as well as the previous failures with transplantation of amniotic membrane, for the treatment of burns, has been mostly abandoned. However, tissue engineering and the substrates used are extremely expensive.

The authors of the present invention have surprisingly found that, by drying the amniotic membrane, a material that is usually discarded, the amniotic membrane is rendered immunologically inert and does not require culture of autologous keratinocytes since the membrane without the epithelium but with the intact ECM allowed the spontaneous colonisation with autologous cells. Based in this finding the method next described has been invented which allows circumventing state of art inconveniences, namely the problems of rejection that occurs with the traditional skin grafts.

### METHOD OF THE INVENTION

As above stated the present invention refers to a method for the preparation of immunologically inert amniotic membranes using foetal membranes from programmed cesareans in sterilized conditions from mothers serum negative for Human Immunodeficiency Virus (HIV), Human Hepatitis B and C Virus (HBV; HCV) and Syphilis.

The method of the invention is by comprises the following steps:

a. Separation of the amnion from the corion and residuals of the decidua,

b. Washing the amnion with the saline solution eliminating any contaminant fragment from the maternal side,

c. Stretching the amnion in a perforated metallic sheet, drying with gauze and covering all the membrane surface with gauze,

d. Covering the gauze with a second perforated metallic sheet and folding the two edges of the sheet, in order to constitute the closed package,

e. Sealing the closed package in a sterilizing sleeve,

f. Maintaining the closed package during 96 hours at the temperature of 37° C. in the atmosphere containing 5% of CO₂.

g. Re-hydrating the membrane in a physiologic solution (0,9% of NaCl).

h. Cryopreserving the membrane in culture medium containing 10% of dimethyl sulphoxyd.

The temperature of the membrane in step f) may be about 4° C., keeping constant the other conditions.

The invention also includes the immunologically inert amniotic membranes obtained by the method of the invention and recovered by re-hydration in a saline solution.

The membranes of the invention find use in the preparation of products aimed at:

Transplantation on skin with burns of the 2_{nd} or 3^{rd} degrees,

regeneration of the peripheral nerves,

cornea graft,

reconstruction of the bladder and urethra,

correction of cardiac malformations such as inter-auricular and inter-ventricular communications,

reconstruction of valvular leaflets.

The saline solution that constitutes the rehydration medium of the amniotic membrane may be RPMI 1640 medium containing 10 mM Hepes and 2 mM L-glutamine.

Experimental Tests

For experimental verification of the lack of immunogenicity of the dried amnion, the following tests have been performed which may not be considered as limitative of the present invention scope.

Test 1—Skin Transplant

Test animals—Six 2.5 month old Wistar rats were used for these experiments.

Experimental Procedure—Full thickness skin fragments of a size of 4×6 cm were excised from the back of the animal and replaced with an equal size fragment of the membrane. Suture was performed with 6/0 silk. Control animals were implanted, using the same procedure, with fresh (viable) membrane from the same donor.

Small fragments of the implanted membrane and the contiguous normal skin were collected at different times after implantation, for histological analysis from both experimental and control animals.

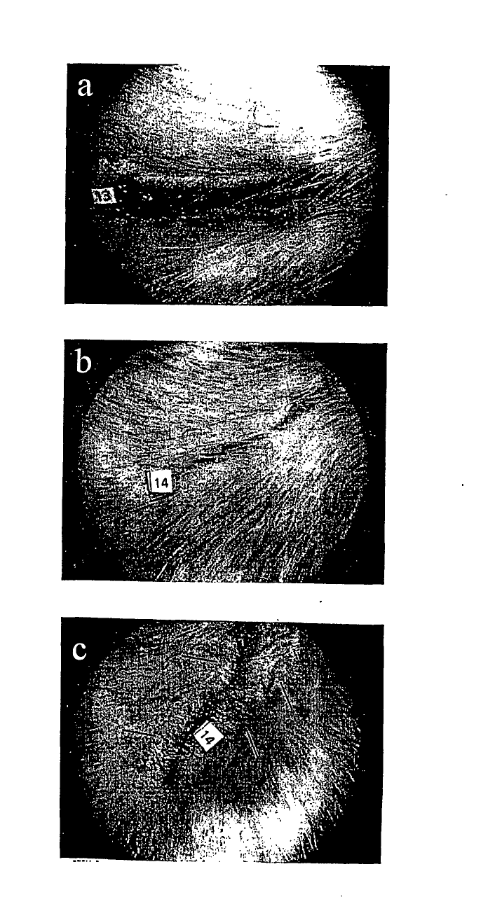

FIG. 1 shows the result of one such tests three weeks after implant. As can bee seen animals implanted with previously dried membrane regenerated skin and fur while those implanted with the viable membrane show an open wound. Furthermore, when the implanted area was shaved in the animal implanted with previously dried membrane the demarcation between normal skin and membrane was still macroscopically visible. Histologically, however, the area corresponding to the membrane could not be distinguished from the normal skin.

Test 2—Nerve regeneration

Test animals—Six 2.5 month old Wistar rats were used for these experiments.

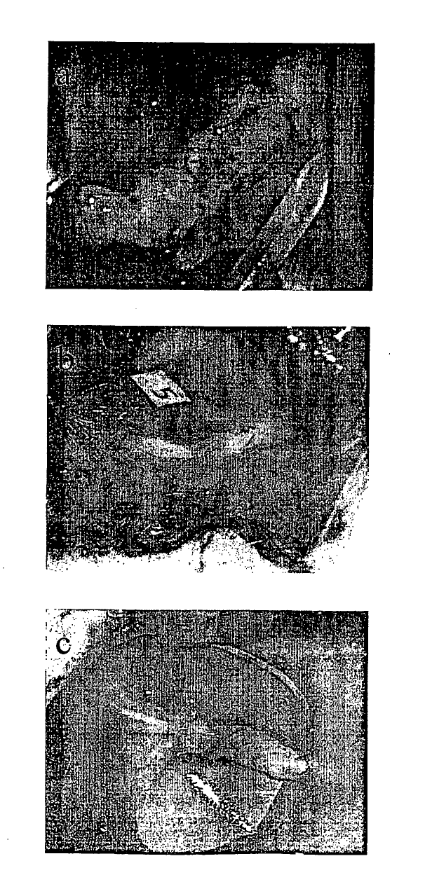

Experimental Procedure—One centimetre of sciatic nerve was excised and replaced with human amniotic membrane tube (FIG. 2).

Morphological and functional evaluation of nerve regeneration, was performed by electrophysiologic and histologic analysis in all animals.

The results obtained, indicate that immunologically inert human amniotic membrane, can promote the regeneration of neurones, stimulates new vascularization, with functional recovery being complete after three to four weeks.

The same type of experiments have been recently reported using amniotic membrane in which the epithelial cells were unintentionally killed by heat. The authors claim that the membrane is reabsorbed¹⁴. It is possible that this experiments worked because they were performed in rats, in which the nerve regeneration time is extremely short. In humans, however, this is not the case and reabsorption of the membrane before total nerve regeneration will eventually arrest the nerve growth. On the other hand this reabsorption is, most likely due to the immune response triggered by the dead cells. This should not occur with the present membrane.

It must be noted that the invention is not limited to the described embodiments, which must be considered as merely illustrative and not limitative of the invention scope which is defined by the attached claims.

### REFERENCES

(1) Wang, H. J.; Cheng, T. M.; Cheng, L. F.; Cheng, T. Y. & Tung, Y. M. (1995) “Human keratinocytes culture using porcine pituitary extract in serum-free medium”; Burns 21 (7), 503-506.

(2) Sheridan, R. L. & Tompkins, R. G. (1999) “Skin substitutes in burns”; Burns 25, 97-103.

(3) Dua, Harminder S. (1999) “Amniotic membrane transplantation”; Br J Ophthalmol 83 (Junho), 748-752.

(4) Lee, S. H. & Tseng S. C. (1997) “Amniotic membrane transplantation for persistent epithelial defects with ulceration”; Am J Ophthalmol 123 (3), 303-12.

(5) Tseng, Scheffer C.C.; Prabhasawat, Pinnita & Lee, Shwu-Huey (1997) “Amniotic membrane transplantation for conjunctival surface reconstruction”; Am J Ophtalmol 124(6), 765-74.

(6) Tseng. Patente norte-americana No. U.S. Pat. No. 6,152,142.

(7) Solomon, Abraham; Pires, Renato T. F. & Tseng, Scheffer C. G. (2001) “Amniotic membrane transplantation after extensive removal of primary and recurrent pterygia”; Ophalmology 108 (3), 449-60.

(8) Meller, Daniel; Maskin, Steven L.; Pires, Renato T. F. & Tseng, Scheffer C. G. (2000) “Amniotic membrane transplantation for symptomatic conjunctivochalasis refractory to medical treatments”; Cornea 19 (6), 796-803.

(9) Shimazaki, Jun; Shinozaki, Naoshi & Tsubota, Kazuo (1998) “Transplantation of amniotic membrane and limbal autograft for patients with recurrent pterygium associated with symblepharon”; Br J Ophalmol 82 (Marco), 235-240

(10) Schwab, Ivan R.; Reyes, Merle & Isseroff, R. Rivakh (2000) “Successful transplantation of bioengineered skin tissue replacements in patients with ocular surface disease”; Cornea 19 (4), 421-426.

(11) Tsay, Ray Jui-Fang, Li, Lien-Min & Chen, Jan-Kan (2000) “Reconstruction of damage corneas by transplantation of autologous limbal epithelial cells”; The New England Journal of Medicine 13, 86-93.

(12) Wright, Kathryn A., Nadire, Kathleen B.; Busto, Patricia; Tubo, Ross; McPherson, John M. & Wentworth, Bruce M. (1998) “Alternative delivery of keratinocytes using the polyurethane membrane and implications for its use in the treatment of full-thickness burn injury”; Burns 24, 7-17.

(13) Eaglstein, William H. & Falanga, Vincent (1998) “Tissue engineering and the development of apligraft the human skin equivalent”, Cutis 62 (15), 1-5.

(14) Mohammad, J., Shenag, J., Rabinovsky, E. and Shennag, S. Modulation of peripheral nerve regeneration: the tissue-engineering approach. The role of Amnion tube nerve conduit across the 1-centimeter nerve gap. Plast. Reconstr. Surg. (2000) 105(2) 660-6.

1. A method for the preparation of immunologically inert amniotic membranes using foetal membranes collected from programmed caesareans under sterile conditions, characterized by comprising the following steps:
a. Separation of the amnion from the corion and residuals of the decidua,
b. Washing the amnion with the saline solution eliminating any contaminant fragment from the maternal side,
c. Stretching the amnion in a perforated metallic sheet, drying with gauze and covering all the membrane surface with gauze,
d. Covering the gauze with a second perforated metallic sheet and folding the two edges of the sheet, in order to constitute the closed package,
e. Sealing the closed package in a sterilizing sleeve,
f. Maintaining the closed package during 96 hours at the temperature of 37° C. in the atmosphere containing 5% of CO₂.
g. Re-hydrating the membrane in a physiologic solution (0,9% of NaCl).
h. Cryopreserving the membrane in culture medium containing 10% of dimethyl sulphoxyd.

2. A method according to claim 1, characterized by, in step f), the temperature for driying the membrane being 4° C., the other conditions being the same.

3. Immunologically inert amniotic membranes, characterized by being obtained using the methods according to claims 1 or 2.

4. Immunologically inert amniotic membranes according to claim 3, characterized by their recovery being performed by re-hydration in a saline solution.

5. Use of the immunologically inert amniotic membranes according to claim 3, characterized by being applied namely to the preparation of a product aimed to be transplanted in the skin with burns of the 2_{nd} or 3^{rd} degree.

6. Use of the immunologically inert amniotic membranes according to claim 3, characterized by being applied namely to the preparation of a long lasting nerve sleeve aimed at the regeneration of the peripheral nerves.

7. Use of the immunologically inert amniotic membranes according to claim 3, characterized by being applied namely to the preparation of a product aimed at cornea graft.

8. Use of the immunologically inert amniotic membranes according to claim 3, characterized by being applied namely to the preparation of a product aimed at reconstruction of the bladder and ureters.

9. Use of the immunologically inert amniotic membranes according to claim 3, characterized by being applied to the correction of cardiac mal-formations such as inter-auricular and inter-ventricular communications.

10. Use of the immunologically inert amniotic membranes according to claim 3, characterized by being applied namely to the preparation of a product aimed at reconstruction of valvular leaflets.
